(19) United States
(12) Patent Application Publication
(10) Pub. No.: US 2009/0010972 A1
(43) Pub. Date: Jan. 8, 2009

(54) DEODORANT COMPOSITIONS

(76) Inventors: Barbara Marie Modafari, Mason, OH (US); David Frederick Swaile, Cincinnati, OH (US)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/047,420

(22) Filed: Mar. 13, 2008

Related U.S. Application Data
(60) Provisional application No. 60/918,496, filed on Mar. 16, 2007.

(51) Int. Cl. A61K 8/06; A61K 8/36; A61Q 15/00

(52) U.S. Cl. 424/401; 424/65

(57) ABSTRACT

Deodorant compositions comprising an emulsion including a continuous polar solvent phase and a discontinuous oil phase are provided. The oil phase contains a water insoluble oil ingredient and a fragrance composition, each of which are chosen such that at least some of the fragrance components preferentially associate with the oil ingredient within the emulsion to provide an improved scent expression profile.

### CROSS REFERENCE TO RELATED APPLICATION

This application claims the benefit under 35 USC 119(e) to U.S. Application Ser. No. 60/918,496, filed Mar. 16, 2007.

### FIELD OF THE INVENTION

The present invention is directed to deodorant compositions comprising an emulsion including a continuous polar solvent phase and a discontinuous oil phase, wherein the oil phase contains a fragrance material.

### BACKGROUND OF THE INVENTION

Deodorant and antiperspirant compositions typically comprise one or more fragrance materials that are intended to mask malodor and/or provide a pleasant scent expression. Various encapsulation and complexing technologies have been developed to delay the expression of some of the fragrance material until a triggering event such as perspiration occurs. These technologies can provide multiple scent expressions or extended scent expression throughout the use period so that a consumer perceives a relatively uniform level of malodor and/or wetness protection during the use period. Known approaches for providing scent longevity however can be difficult to leverage within polar matrices since the polar components themselves can prematurely release the fragrance material prior to the onset of perspiration. Accordingly, there is a need for a scent-releasing technology that can provide scent longevity out of polar deodorant compositions.

### SUMMARY OF THE INVENTION

The present invention is generally directed to deodorant compositions comprising an emulsion including a continuous polar solvent phase and a discontinuous oil phase. The oil phase may contain a relatively low to moderate polarity (high Clog P) oil material and a fragrance composition that comprises one or more high Clog P fragrance components. Without being bound by theory, it is believed that these fragrance components are substantially more soluble in the low to moderately polar high Clog P oil than the continuous polar phase, and thereby “trapped” or held within the emulsion such that the composition may produce a reduced fragrance strength impact when the composition is smelled at shelf or immediately at application to the body. As the polar solvent evaporates or is otherwise removed from the deodorant product film applied to the body, the fragrance material can release from the oil phase to provide an odor masking or fragrance benefit to the consumer.

### DETAILED DESCRIPTION OF THE INVENTION

The present invention may be understood more readily by reference to the following detailed description of illustrative and preferred embodiments. It is to be understood that the scope of the claims is not limited to the specific features, methods, conditions, or parameters described herein, and that the terminology used herein is for the purpose of describing particular embodiments by way of example only and it not intended to be limiting of the claimed invention. Also, as used in the specification including the appended claims, the singular forms “a,” “an,” and “the” include the plural, and reference to a particular numerical value includes at least that particular value, unless the context clearly dictates otherwise. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

All percentages, parts and ratios are based upon the total weight of the compositions of the present invention, unless otherwise specified. All such weights as they pertain to listed ingredients are based on the active level and, therefore, do not include solvents or by-products that may be included in commercially available materials, unless otherwise specified. The term “weight percent” may be denoted as “wt. %” herein.

As used herein, “comprising” means that other steps and other ingredients which do not affect the end result can be added. This term encompasses the terms “consisting of” and “consisting essentially of”. The compositions of the present invention can comprise, consist of, and consist essentially of the elements and limitations of the invention described herein, as well as any of the additional or optional ingredients, components, steps, or limitations described herein.

As used herein, the term “deodorant composition,” includes personal care compositions that comprise fragrance materials that are intended to mask malodor and/or provide a fragrance expression to consumers. The deodorant compositions may also comprise a deodorant active and /or an antiperspirant active. The deodorant compositions may take on a variety of product forms, including, but not limited to, solid sticks, soft solids, creams, body sprays, aerosols, and roll-ons.

The use of Clog P values is well known in the chemical arts as a calculated value that represents the relative affinity that a material has for partitioning between octanol and water, so that a material that partitions more readily into octanol would tend to be more lipophillic and have a higher Clog P value than a material that partitions less readily into octanol. For purposes of defining the deodorant compositions of the present invention or ingredients thereof, Clog P values can be obtained from or calculated by the methods described in Handbook of Physical Properties of Organic Chemicals, Edited by Philip H. Howard and William M. Meylan, CRC Press-Lewis Publishers, 1997.

Clog P values can also be determined by the Pamona Med Chem/Daylight “C LOG P” program, Version 4.42, available from Biobyte Corporation, Claremont, Calif. Other suitable methods for determining Clog P values include the fragment approach described by Hansch and Leo (cf., A. Leo, in Comprehensive Medicinal Chemistry, Vol. 4, C. Hansch, P. G. Sammens, J. B. Taylor and C. A. Ramsden, Eds., p. 295, Pergamon Press, 1990), which description is incorporated herein by reference. Still other suitable methods are described or provided by Daylight Information Systems, Mission Viejo, Calif., Daylight V4.61, Algorithm: V3.05, Database: V16. General information pertaining to Clog P values and methodologies are described in Chemical Reviews, 93(4), 1993, 1281-1306. As used herein, Clog P values include calculated and measured log P values.

As used herein, the term “high Clog P” includes Clog P values that are greater than 3, greater than 4, greater than 5, and greater than 6.

Deodorant compositions of the present invention include a continuous polar solvent phase and a discontinuous oil phase dispersed therein. The oil phase typically includes a hydrocarbon component, a fragrance composition, and one or more surfactants to permit formation of an emulsion during the manufacture of the deodorant compositions. These features and ingredients will be discussed below, followed by a description of optional ingredients that may or may not be employed in the deodorant compositions of the present invention.

### I. Polar Solvent Phase

Exemplary deodorant compositions comprise a polar solvent phase that includes one or more polar solvents. A representative, non-limiting, list of suitable polar solvents includes water, ethanol, propylene glycol, dipropylene glycol, tripropylene glycol, glycerin, methyl propylene glycol, butylene glycol, polyethylene glycol, polypropylene glycol, and mixtures thereof. Additional polar solvents known to the skilled artisan may also be employed. The polar solvents may be employed at a concentration level of from about 10% to about 90%, by weight of the composition, and preferably at a concentration level of from about 60% to about 85%, by weight of the composition. Other concentration levels may also be used.

The deodorant compositions of the present invention may optionally comprise thickening agents within the polar solvent phase. The thickening agents can help provide the composition with the desired viscosity, rheology, texture and/or product hardness, or to otherwise help suspend any dispersed solids or liquids within the composition. The term “thickening agent” may include any material known or otherwise effective in providing suspending, gelling, viscosifying, solidifying or thickening properties to the composition or which otherwise provide structure to the final product form. These thickening agents may include gelling agents, polymeric or nonpolymeric agents, inorganic thickening agents, or viscosifying agents. The thickening agents may include organic solids, silicone solids, crystalline or other gellants, inorganic particulates such as clays or silicas, or combinations thereof.

The concentration and type of the thickening agent selected for use in compositions of the present invention will vary depending upon the desired product form, viscosity, and hardness. The thickening agents suitable for use herein, may have a concentration range from at least about 0.1%, at least about 3%, or at least about 5%, but no more than about 35%, no more than about 20%, or no more than about 10%, by weight of the composition.

Non-limiting examples of suitable gelling agents include fatty acid gellants, salts of fatty acids, hydroxyl acids, hydroxyl acid gellants, esters and amides of fatty acid or hydroxyl fatty acid gellants, cholesterolic materials, dibenzylidene alditols, lanolinolic materials, fatty alcohols, triglycerides, sucrose esters such as SEFA behenate, inorganic materials such as clays or silicas, other amide or polyamide gellants, and mixtures thereof.

Suitable gelling agents include fatty acid gellants such as fatty acid and hydroxyl or alpha hydroxyl fatty acids, having from about 10 to about 40 carbon atoms, and ester and amides of such gelling agents. Non-limiting examples of such gelling agents include, but are not limited to, 12-hydroxystearic acid, 12-hydroxylauric acid, 16-hydroxyhexadecanoic acid, behenic acid, eurcic acid, stearic acid, caprylic acid, lauric acid, isostearic acid, and combinations thereof. Preferred gelling agents are 12-hydroxystearic acid, esters of 12-hydroxystearic acid, amides of 12-hydroxystearic acid and combinations thereof.

Other suitable gelling agents include amide gellants such as disubstituted or branched monoamide gellants, monsubstituted or branched diamide gellants, triamide gellants, and combinations thereof, including n-acyl amino acid derivatives such as n-acyl amino acid amides, n-acyl amino acid esters prepared from glutamic acid, lysine, glutamine, aspartic acid, and combinations thereof. Other suitable amide gelling agents are described in U.S. Pat. No. 5,429,816, issued Jul. 4, 1995, and U.S. Pat. No. 5,840,287, filed Dec. 20, 1996.

Still other examples of suitable gelling agents include fatty alcohols having at least about 8 carbon atoms, at least about 12 carbon atoms but no more than about 40 carbon atoms, no more than about 30 carbon atoms, or no more than about 18 carbon atoms. For example, fatty alcohols include but are not limited to cetyl alcohol, myristyl alcohol, stearyl alcohol and combinations thereof.

Non limiting examples of suitable tryiglyceride gellants include tristearin, hydrogenated vegetable oil, trihydroxysterin (Thixcin® R, available from Rheox, Inc.), rape seed oil, castor wax, fish oils, tripalmitin, Syncrowax® HRC and Syncrowax® HGL-C (Syncrowax® available from Croda, Inc.).

### II. Oil Phase

Exemplary deodorant compositions also comprise an oil phase. The oil phase may contain a variety of different water insoluble oil ingredients, including, for example, organic liquid carriers such as mineral oil, petrolatum, isohexadecane, isododecane, various other hydrocarbon oils, and mixtures thereof. Specific non-limiting examples of suitable branched chain hydrocarbon oils include isoparaffins available from Exxon Chemical Company as Isopar C™ (C7-C8 Isoparaffin), Isopar E™ (C8-C9 Isoparaffin), Isopar GT (C10-11 Isoparaffin), Isopar H™ (C11-C12 Isoparaffin), Isopar L™ (C11-C13 Isoparaffin), Isopar M™ (C13-C14 Isoparaffin), and combinations thereof. Other non-limiting examples of suitable branched chain hydrocarbons include Permethyl 99A (isododecane), Permethyl 102A (isoeicosane), Permethyl™ 101A (isohexadecane), and combinations thereof. The Permethyl series are available from Preperse, Inc., South Plainfield, N.J., U.S.A. Other non-limiting examples of suitable branched chain hydrocarbons include petroleum distillates such as those available from Phillips Chemical as Soltrol™ 130, Soltrol™ 170, and those available from Shell as Shell Sol™ 70, −71, and −2033, and mixtures thereof.

Examples of other suitable organic liquid carriers include the Norpar series of paraffins available from Exxon Chemical Company as Norpar 12, −13, and −15; octyldodecanol; butyl stearate; diisopropyl adipate; dodecane; octane; decane; C1-C15 alkanes/cycloalkanes available from Exxon as Exxsol™ D80; C12-C15 alkyl benzoates available as Finsolv-TN™ from Finetex; and mixtures thereof. Other suitable liquid carriers include benzoate co-solvents, cinnamate esters, secondary alcohols, benzyl acetate, phenyl alkane, and combinations thereof.

One preferred list of oil ingredients includes the following hydrocarbon, high Clog P liquids: mineral oil, petrolatum, isoparraffins, PPG-14 butyl ether, isopropyl myristate, butyl stearate, cetyl ocatnoate, butyl myristate, C12-C15 alkyl benzoate (e.g., Finsolv-TN™ from Finetex), octyldodecnaol, isostearyl isostearate, octododecyl benzoate, isostearyl lactate, isostearyl palmitate, and isobutyl stearate.

The oil phase also includes a fragrance composition that comprises one or more high Clog P fragrance components. The fragrance composition within the oil phase may be employed at a concentration level of from about 0.1% to about 10%, by weight of the composition, preferably from about 0.1% to about 5%. Other concentration levels however are contemplated by the present invention. Table 1 below provides an exemplary list of such fragrance components.

TABLE 1
Exemplary Fragrance Component List

| | Clog P |
|---|---|
| Fragrance Component | value |
| Allyl cyclohexane propionate | 3.935 |
| Ambrettolide | 6.261 |
| Amyl benzoate | 3.417 |
| Amyl cinnamate | 3.771 |
| Amyl cinnamic aldehyde | 4.324 |
| Amyl cinnamic aldehyde dimethyl acetal | 4.033 |
| Iso-amyl salicylate | 4.601 |
| Aurantiol (Trade name for Hydroxycitronellal- | 4.216 |
| methylanthranilate) | |
| Benzyl salicylate | 4.383 |
| para-tert-Butyl cyclohexyl acetate | 4.019 |
| Iso butyl quinoline | 4.193 |
| beta-Caryophyllene | 6.333 |
| Cadinene | 7.346 |
| Cedrol | 4.530 |
| Cedryl acetate | 5.436 |
| Cedryl formate | 5.070 |
| Cinnamyl cinnamate | 5.480 |
| Cyclohexyl salicylate | 5.265 |
| Cyclamen aldehyde | 3.680 |
| Diphenyl methane | 4.059 |
| Diphenyl oxide | 4.240 |
| Dodecalactone | 4.359 |
| Iso E Super (Trade name for 1-(1,2,3,4,5,6,7,8-Octahydro- | 3.455 |
| 2,3,8,8-tetramethyl-2-naphthalenyl)-ethanone) | |
| Ethylene brassylate | 4.554 |
| Ethyl undecylenate | 4.888 |
| Exaltolide (Trade name for 15-Hydroxyentadecanloic acid, | 5.346 |
| lactone) | |
| Galaxolide (Trade name for 1,3,4,6,7,8-Hexahydro-4,6,6,7,8,8- | 5.482 |
| hexamethylcyclopenta-gamma-2-benzopyran) | |
| Geranyl anthranilate | 4.216 |
| Geranyl phenyl acetate | 5.233 |
| Hexadecanolide | 6.805 |
| Hexenyl salicylate | 4.716 |
| Hexyl cinnamic aldehyde | 5.473 |
| Hexyl salicylate | 5.260 |
| Alpha-Irone | 3.820 |
| Lilial (Trade name for para-tertiary-Butyl-alpha-methyl | 3.858 |
| hydrocinnamic aldehyde) | |
| Linalyl benzoate | 5.233 |
| Methyl dihydrojasmone | 4.843 |
| Gamma-n-Methyl ionone | 4.309 |
| Musk indanone | 5.458 |
| Musk tibetine | 3.831 |
| Oxahexadecanolide-10 | 4.336 |
| Oxahexadecanolide-11 | 4.336 |
| Patchouli alcohol | 4.530 |
| Phantolide (Trade name for 5-Acetyl-1,1,2,3,3,6-hexamethyl | 5.977 |
| indan) | |
| Phenyl ethyl benzoate | 4.058 |
| Phenylethylphenylacetate | 3.767 |
| Phenyl heptanol | 3.478 |
| Alpha-Santalol | 3.800 |
| Thibetolide (Trade name for 15-Hydroxypentadecanoic acid, | 6.246 |
| lactone) | |
| Delta-Undecalactone | 3.830 |
| Gamma-Undecalactone | 4.140 |
| Vetiveryl acetate | 4.882 |
| Ylangene | 6.268 |

The fragrance composition within the oil phase may contain one of the fragrance materials included in Table 1, or a combination of the listed materials. The fragrance composition may also contain fragrance components other than those listed in Table 1, with preferred fragrance components being those that have a Clog P value that is greater than 3.

In accordance with some of the preferred embodiments, at least about 25%, 50%, 60%, or 80%, by weight, of the fragrance composition within the oil phase comprises fragrance components that have a Clog P value greater than about 3, greater than about 4, or greater than about 5. In accordance with another preferred embodiment, at least about 25%, by weight, of the fragrance composition within the oil phase comprises fragrance components that have a boiling point of greater than about 250° C.

Without being bound by theory, it is believed that fragrance materials having relatively high Clog P values will preferentially associate with the oil ingredient (also having a relatively high Clog P value), and thereby be “trapped” or held within the discontinuous oil phase of the deodorant composition. The trapped or held fragrance component(s) can then later be released after the polar solvents evaporate or are otherwise removed from the deodorant product film applied to the body. Thus, applicants have discovered that matching oil ingredients with fragrance components according to their Clog P is one technique that may provide scent longevity out of polar deodorant compositions. In accordance with this theory and some of the preferred embodiments, at least 25% or 50%, by weight, of the fragrance composition within the oil phase comprises fragrance components that have a Clog P value that is within 3 units or within 2 units of the Clog P value of an oil ingredient also within the oil phase.

### III. Surfactant

Exemplary deodorant compositions also comprise one or more surfactants to facilitate formation of the oil/polar solvent emulsion. The skilled artisan would readily appreciate that numerous surfactants may be employed to accomplish this function. A representative, non-limiting list of surfactants includes steareth-2, steareth-21, stearyl alcohol, C20-C40 alcohol, C20-C40 pareth 40, and mixtures thereof.

### IV. Optional Ingredients

The deodorant compositions of the present invention may comprise a variety of optional ingredients. For example, deodorant actives and/or antiperspirant actives may be employed. A representative, non-limiting list of deodorant actives includes antimicrobial agents (e.g., bacteriocides, fungicides), malodor-absorbing material, and combinations thereof. For example, antimicrobial agents may comprise cetyl-trimethylammonium bromide, cetyl pyridinium chloride, benzethonium chloride, diisobutyl phenoxy ethoxy ethyl dimethyl benzyl ammonium chloride, sodium N-lauryl sarcosine, sodium N-palmethyl sarcosine, lauroyl sarcosine, N-myristoyl glycine, potassium N-lauryl sarcosine, trimethyl ammonium chloride, sodium aluminum chlorohydroxy lactate, triethyl citrate, tricetylmethyl ammonium chloride, 2,4,4′-trichloro-2′-hydroxy diphenyl ether (triclosan), 3,4,4′-trichlorocarbanilide (triclocarban), diaminoalkyl amides such as L-lysine hexadecyl amide, heavy metal salts of citrate, salicylate, and piroctose, especially zinc salts, and acids thereof, heavy metal salts of pyrithione, especially zinc pyrithione, zinc phenolsulfate, farnesol, and combinations thereof.

One suitable class of antiperspirant active includes astringent metallic salts, especially inorganic and organic salts of aluminum, zirconium and zinc, as well as mixtures thereof. Particular antiperspirant active examples include, but are not limited to, aluminum-containing and/or zirconium-containing salts or materials, such as aluminum halides, aluminum chlorohydrate, aluminum hydroxyhalides, zirconyl oxyhalides, zirconyl hydroxyhalides, and mixtures thereof.

Aluminum salts useful in the present invention include those that conform to the formula:

Al₂(OH)ₐCl_{b}.xH₂O

wherein a is from about 0 to about 5; the sum of a and b is about 6; x is from about 1 to about 8; where a, b, and x may have non-integer values. For example, aluminum chlorohydroxides referred to as “3/4 basic chlorohydroxide,” wherein a is about 4.5; “5/6 basic chlorohydroxide”, wherein a=5; and “2/3 basic chlorohydroxide”, wherein a=4 may be used. Processes for preparing aluminum salts are disclosed in U.S. Pat. No. 3,887,692, issued to Gilman on Jun. 3, 1975; U.S. Pat. No. 3,904,741, issued to Jones et al. on Sep. 9, 1975; and U.S. Pat. No. 4,359,456 issued to Gosling et al. on Nov. 16, 1982. A general description of these aluminum salts can also be found in Antiperspirants and Deodorants, Cosmetic Science and Technology Series Vol. 20, 2nd edition, edited by Karl Laden. Mixtures of aluminum salts are described in British Patent Specification 1,347,950, filed in the name of Shin et al. and published Feb. 24, 1974.

Zirconium salts that may be employed in the present invention include those which conform to the formula:

ZrO(OH)₂₋ₐClₐ.xH₂O

wherein a is from about 0.5 to about 2; x is from about 1 to about 7; where a and x may both have non-integer values. These zirconium salts are described in Belgian Patent 825,146, issued to Schmitz on Aug. 4, 1975. Useful to the present invention are zirconium salt complexes that additionally contain aluminum and glycine, commonly known as “ZAG complexes”. These complexes contain aluminum chlorohydroxide and zirconyl hydroxy chloride conforming to the above-described formulas. Such ZAG complexes are described in U.S. Pat. No. 4,331,609, issued to Orr on May 25, 1982 and U.S. Pat. No. 4,120,948, issued to Shelton on Oct. 17, 1978.

The deodorant compositions may optionally comprise dyes, pigments, or other colorants. The compositions may also include additional fragrance materials beyond those described above and that are initially held within the emulsion. The compositions may comprise pH buffers (e.g., tetra sodium EDTA) or other formulation tools. Nonlimiting examples of other optional ingredients include emollients, humectants, soothing agents, medicaments, baking soda and related materials, preservatives, and soothing agents such as aloe vera, allantoin, D-panthenol, pantothenic acid derivatives, avocado oil and other vegetative oils, and lichen extract.

### V. EXAMPLES

The following examples further describe and demonstrate embodiments within the scope of the present invention. The examples are given solely for the purpose of illustration and are not to be construed as limitations of the present invention as many variations thereof are possible without departing from the spirit and scope of the invention.

Deodorant Composition Examples A-E. The numerical values in the table are in percent by weight of the total composition.

| Ingredient | A | B | C | D | E |
|---|---|---|---|---|---|
| dipropylene glycol | 45 | 30 | 20 | 45 | 20 |
| propylene glycol | 22 | | 22 | 22 | |
| tripopylene glycol | | | 25 | | |
| glycerine | | 10 | | | |
| PEG-8 | | 20 | | | |
| water | QS | QS | QS | QS | QS |
| 50% aqueous aluminum | | | | | 40 |
| chlorohydrate | | | | | |
| sodium stearate | 5.5 | 5.5 | 5.5 | 5.5 | |
| tetra sodium EDTA | 0.05 | 0.05 | 0.05 | 0.05 | |
| steareth-2 | 2.2 | | | | |
| steareth-21 | 1.0 | 1.5 | | | |
| stearyl alcohol | | 2.0 | | 2.0 | |
| C20-C40 alcohol ^{1.} | | | 2.0 | | 2.0 |
| C20-C40 pareth 40 ^{2.} | | | 1.0 | 1.0 | 1.0 |
| petrolatum | 1.75 | | 1.75 | 1.75 | 1.75 |
| mineral oil | | 1.75 | | | |
| dye | 0.002 | 0.002 | 0.002 | 0.002 | |
| high C logP fragrance | 0.2-2 | 0.2-2 | 0.2-2 | 0.2-2 | 0.2-2 |
| composition^{3.} | | | | | |
| base fragrance | 0.5-2 | 0.5-2 | 0.5-2 | 0.5-2 | 0.5-2 |

QS-indicates that this material is used to bring the total to 100%.
^{1.} Available from New Phase Technologies as Performachol 350.
^{2.} Available from New Phase Technologies as Performathox 480.
^{3.}This fragrance comprises at least 50% fragrance components with C log P values greater than 3

Examples A and B can be made by a dual stream process as follows:
Hot Stream Create an oil phase by combining steareth-2 or stearyl alcohol, steareth-21, petrolatum, and the high Clog P fragrance composition in a container and heat to about 65° C. Create an aqueous phase by combining the water, sodium stearate, EDTA, and about 10% of the glycols in a separate container and heating to about 85° C. Cool the aqueous solution to about 75° C. Once the oil phase has melted and is homogenous, add it to the aqueous phase with agitation.
Cold Stream Combine the remaining polar emollients, base fragrance, and dye in a separate container and mix until homogenous.
Combine the hot stream and the cold stream via a two-stream filler, at approximately a 1:1 ratio and mix (e.g., with a static mixer) to form the deodorant composition. Charge the deodorant composition into a suitable container and cool.
Examples C and D can be made as follows: Create an oil phase by combining the C20-40 alcohol or stearyl alcohol, C20-C40 pareth 40, petrolatum, and the high Clog P fragrance composition in a container and heat to about 85° C. In a separate container, heat about half of the water to about 85° C. Once the oil phase is melted and homogenous, slowly add it to the heated water and agitate the mixture to form an emulsion. Allow the emulsion to cool slowly to ambient temperature while stirring. Allow the emulsion to equilibrate for at least 6 hours, and preferably, for at least 24 hours. Combine the glycols, the remaining water, sodium stearate, EDTA, and dye in a suitable container and heat to about 85° C. Once the solids are dissolved, add the base fragrance and the emulsion to the solution with agitation. Cool the solution to about 70° C. and pour into an appropriate container.
Example E can be made as follows: Create an oil phase by combining the C20-40 alcohol or stearyl alcohol, C20-C40 pareth 40, petrolatum, and the high Clog P fragrance composition in a container and heat to about 85° C. In a separate container, heat about half of the water to about 85° C. Once the oil phase is melted and homogenous, add it to the water with agitation to form an emulsion. Cool the emulsion to ambient temperature while stirring. Allow the emulsion to equilibrate for at least 6 hours, and preferably, for at least 24 hours. Combine the dipropylene glycol, base fragrance, and the aqueous solution of aluminum chlorohydrate in a suitable container and mix until homogenous liquid is formed. Add the emulsion to this solution and mix until uniform. Pour this product into a suitable container.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A deodorant composition, comprising an emulsion including a polar solvent phase and an oil phase disposed therein, the oil phase comprises a water insoluble oil ingredient having a Clog P value greater than about 3 and a fragrance composition, wherein at least 25%, by weight, of the fragrance composition comprises fragrance components that have a Clog P value greater than about 3.

2. The composition of claim 1, wherein the polar solvent phase comprises water, ethanol, propylene glycol, dipropylene glycol, tripropylene glycol, glycerin, methyl propylene glycol, butylene glycol, polyethylene glycol or polypropylene glycol or mixtures thereof.

3. The composition of claim 1, wherein the water insoluble oil ingredient is selected from the group consisting of mineral oil, petrolatum, isoparraffins, PPG-14 butyl ether, isopropyl myristate, butyl stearate, cetyl ocatnoate, butyl myristate, C12-C15 alkyl benzoate, octyldodecnaol, isostearyl isostearate, octododecyl benzoate, isostearyl lactate, isostearyl palmitate, isobutyl stearate, and mixtures thereof.

4. The composition of claim 1, wherein the water insoluble oil ingredient comprises petrolatum.

5. The composition of claim 1, wherein the water insoluble oil ingredient comprises mineral oil.

6. The composition of claim 1, wherein the water insoluble oil ingredient has a Clog P value of greater than about 4.

7. The composition of claim 1, wherein the water insoluble oil ingredient has a Clog P value of greater than about 5.

8. The composition of claim 1, wherein at least 50%, by weight, of the fragrance composition comprises fragrance components that have a Clog P value greater than about 3.

9. The composition of claim 1, wherein at least 60%, by weight, of the fragrance composition comprises fragrance components that have a Clog P value greater than about 3.

10. The composition of claim 1, wherein at least 80%, by weight, of the fragrance composition comprises fragrance components that have a Clog P value greater than about 3.

11. The composition of claim 1, wherein at least 25%, by weight, of the fragrance composition comprises fragrance components that have a Clog P value greater than about 4.

12. The composition of claim 1, wherein at least 50%, by weight, of the fragrance composition comprises fragrance components that have a Clog P value greater than about 4.

13. The composition of claim 1, wherein at least 25%, by weight, of the fragrance composition comprises fragrance components that have a Clog P value greater than about 5.

14. The composition of claim 1, wherein at least 25%, by weight, of the fragrance composition comprises fragrance components that have a boiling point of greater than about 250° C.

15. The composition of claim 1, wherein the composition is in the form of a solid or cream.

16. The composition of claim 1, further comprising an antiperspirant and/or deodorant active.
